(19) United States
(12) Patent Application Publication
(10) Pub. No.: US 2023/0263725 A1
(43) Pub. Date: Aug. 24, 2023

(54) INTRANASAL ADMINISTRATION OF AN ANTIOXIDANT COMPOUND FOR TREATING CORONAVIRUS INFECTION

(71) Applicant: Beyond Barriers Therapeutics, Inc., Glencoe, IL (US)

(72) Inventors: Ryan Kole, Glencoe, IL (US); Tom De Gregoris, Chicago, IL (US)

(21) Appl. No.: 18/015,316

(22) PCT Filed: Jul. 9, 2021
(86) PCT No.: PCT/US2021/041163

§ 371 Date: Jan. 9, 2023

Related U.S. Application Data
(60) Provisional application No. 63/049,905, filed on Jul. 9, 2020.

(51) Int. Cl. A61K 9/00; A61K 45/06; A61K 47/40; A61K 31/198; A61P 31/14

(52) U.S. Cl. CPC A61K 9/0043; A61K 31/198; A61K 45/06; A61K 47/40; A61P 31/14

(57) ABSTRACT

Infection by SARS-CoV-2 is impacting human subjects in their respiratory system and in some cases, their central nervous system (CNS). The virus is hurting certain groups of people more than others, such as the elderly and those with other pre-existing illnesses. Both groups are believed to have lower levels of the antioxidant, glutathione. By delivery of N-acetyl cysteine (NAC) to increase glutathione levels, the present methods reduce the severity of the virus and its symptoms concentrating on the brain and the lungs. NAC may also prevent mild COVID-19 cases from becoming severe. NAC, whether delivered intranasally, inhaled through the mouth, orally or other ways may act as a therapeutic for this virus.

### CROSS-REFERENCE TO RELATED APPLICATIONS

This application claims priority to and benefit of U.S. Provisional Pat. Application No. 63/049,905, filed on Jul. 9, 2020, the contents of which are incorporated herein by reference in their entirety.

### BACKGROUND

COVID-19, the disease caused by the novel coronavirus SARS-CoV-2 has led to hundreds of thousands of deaths worldwide in the first half of 2020, with approximately one-quarter of the reported deaths in the US. With no vaccine or fully effective therapeutic discovered yet and flu season fast approaching in the Northern hemisphere, people are instructed to use social distancing, practice proper hygiene, wear a mask, avoid large crowds and quarantine.

As research continues for vaccines and therapeutics, scientists and doctors are learning more about the disease. It was originally thought to only impact lung functionality. Recently, it has been observed that this infection is also is affecting the central nervous system (CNS). Human coronaviruses are not limited to the respiratory tract; they can also attack the CNS. Recent retrospective and prospective studies of COVID-19 admitted patients reveal that the patients frequently showed neurological manifestations including stroke, dizziness, headache, seizures, impaired consciousness, acute cerebrovascular problems, cerebral hemorrhage and confusion. The loss of consciousness may be due to due to a number of mechanisms such as direct brain infection and injury, toxic-metabolic encephalopathy, and demyelinating disease. There have also been reports of patients experiencing anosmia (loss of sense of smell) and/or ageusia (loss of sense of taste).

Deaths due to COVID-19 are more common in the elderly and those with prior health conditions. These groups of people usually have one thing in common: less endogenous glutathione. Glutathione (GSH) is one of the body’s most important antioxidants that reduce oxidative stress by attacking reactive oxygen species (ROS). It is a tripeptide consisting of cysteine, glycine and glutamic acid. With low levels of GSH, there are increased levels of ROS which could lead to apoptosis.

Some studies have shown that the severity of COVID-19 was correlated to the levels of GSH.

MUCOMYST, a trade name or brand name for an acetylcysteine solution, is for inhalation (mucolytic agent) or oral administration (acetaminophen antidote). MUCOMYST is indicated as adjuvant therapy for patients with abnormal, viscid, or inspissated mucous secretions.

### SUMMARY

As one aspect, the present invention relates to providing NAC or other antioxidant compounds to reduce the symptoms in the CNS, lungs or both caused by SARS-CoV-2. The delivery of the antioxidant compounds could be intranasal delivery, inhaled through the mouth (for example with a nebulizer), intracranially or orally depending on the severity of the case and where the infection is located. The administration can also be used prophylactically by high-risk groups to COVID-19, such as the elderly or those with prior health conditions. The application can even be used by healthy individuals to prevent infection.

Treating the CNS is challenging because the difficulty of drugs getting past the blood brain barrier. Consequently, nasal administration may be an especially beneficial method for treating symptoms in the CNS due the direct nose-to-brain path. However, NAC usually has a strong, unfavorable smell when its used. Therefore, as one aspect of the present invention, a vial or other container can be configured to prevent oxygen exposure, thereby reducing the unpleasant smell of NAC while at the same time possibly increasing the shelf-life. The vial could also work with a nasal delivery device that also allows for delivery without mixing oxygen with NAC or other antioxidants, preventing oxidation.

### BRIEF DESCRIPTION OF THE FIGURES

FIG. 1 shows MR spectroscopy images obtained as described in Example 2.

FIG. 2 shows times of spectroscopic imaging in Study Period 1 of Example 2.

FIGS. 3 and 4 show results from Study Period 1 of Example 2.

FIG. 5 shows times of spectroscopic imaging in Study Period 2 of Example 2.

FIG. 6 shows results from Study Period 2 of Example 2.

FIG. 7 shows combined results from both study periods of Example 2.

FIG. 8 shows that intranasal NAC leads to a substantial increase (>50%) in brain GSH levels.

### DETAILED DESCRIPTION

The present invention relates to a treatment (a reduction or prevention of symptoms, in particular in the brain and lungs) and/or prophylactic for at risk people or the general population by providing N-acetyl cysteine (NAC) or other antioxidant compound orally, intranasally, via inhalation through the mouth or parenterally to increase GSH levels. NAC can increase the GSH levels in the immune system to attack the infections. Throughout this disclosure, references to NAC are representative of antioxidant compounds more generally, and the present disclosure should be understood as describing uses of other antioxidant compounds when it is describing uses of NAC. Further, it is contemplated that antioxidant compounds can be provided as pharmaceutically acceptable salts, so references to antioxidant compounds throughout this disclosure should be understood as also referring to or pharmaceutically acceptable salts thereof.

NAC is an FDA approved drug with a 40- year safety record. NAC is as an FDA approved drug used to treat acetaminophen (i.e., Tylenol®, an anti-inflammatory medication) overdose which can affect the liver. NAC, commonly referred to as the brand name, MUCOMYST, is also approved for use on patients with cystic fibrosis (CF) and chronic obstructive pulmonary disease (COPD). NAC helps loosen the thick mucus in the lungs for those with these two lung illnesses making it easier to breath. It is estimated that about 20% of COVID-19 patients have pneumonia-like symptoms which leads to the air sacs in the lungs getting filled with fluid. This leads to a decrease in the ability to take in oxygen, causing shortness of breath and a cough. Some reports have shown about one-third of people with COVID-19 have sputum production (also referred to as phlegm) which is a thick mucus that is coughed up from the lungs.

While the present invention is not limited to theory, it is expected that NAC can help loosen up the sputum for those experiencing respiratory problems from COVID-19, particularly for severe cases of COVID-19.

The present disclosure provides positive preliminary data showing oral NAC helps COVID-19 patients, and PBPK analysis predicts intranasal NAC to have roughly 9x more bioavailability than oral NAC. One objective of the present technology is to increase GSH levels in COVID-19 patients as soon as possible. The present disclosure also shows that intranasal administration of NAC in healthy humans increases GSH levels in the brain. It is expected that intranasal NAC should be more beneficial than oral NAC, while being similar in cost and ease of use, and without requiring hospitalization. As another advantage, intranasal delivery of NAC is likely to have fewer side effects than orally delivered NAC.

It is also contemplated that NAC could be administered by inhalation through the mouth to get direct access to the lungs for the upper respiratory effects of viral infections such as COVID-19. Nonetheless intranasal administration may have advantages over oral inhalation, as IN administration often leads to some of the therapeutic dripping to the back of the throat where it may provide benefits such as: 1. Acting quickly and being more systemic, 2. Increasing GSH in the brain to protect it from any damage or further damage, such as by attacking the ROS and RNS and 3. Providing some benefit to the lungs when the therapeutic drips down. Intranasal NAC’s high bioavailability also will allow for greater concentration of the therapeutic in the blood circulation, reaching the lungs.

N-Acetylcysteine’s therapeutic benefit may be various viruses, beyond just the coronavirus. In Zhongcheng Shi and Carlos A Puyo’s work, N-Acetylcysteine to Combat COVID-19: An Evidence Review, published Nov. 2nd 2020 in Therapeutics and Clinical Risk Management, they suggested that cellular immunity is necessary to fight a viral infection. This immunity is regulated by an oxidant-antioxidant (includes glutathione (GSH)) balance. As noted earlier, NAC given as a therapeutic leads to increased GSH, helping with this balance. In the elderly and those with weakened immune systems, there is a noticeable decrease in GSH leading to an increase in Reactive Oxygen Species (ROS). This leads to the immune system not working at its full capacity (for example T cell-mediated functions) which may be the cause of increased mortality in the elderly from infectious diseases such as pneumonia.

The addition of NAC can help strengthen the immune system, stop viral replication and reduce inflammation allowing the body to fight off the infection. NAC has also shown an ability to inhibit NF-κB, which may prevent RNA viruses’ ability to replicate.

In a study by S De Flora, C Grassi and L Carati titled Attenuation of influenza-like symptomatology and improvement of cell-mediated immunity with long-term N-acetylcysteine treatment, published in the European Respiratory Journal in July 1997, subjects were given oral NAC over a period of 6 months. The results showed a significant decrease in the frequency of influenza-like symptoms, severity and time confined to a bed. In the NAC treated group, 25% developed a symptomatic form of the A/H1N1 Singapore 6/86 influenza virus versus 79% who received the control.

Accordingly, as another aspect of the present disclosure, methods are provided for treating a disease or disorder associated with a virus infection. In some embodiments, the virus infection is an RNA virus. In some embodiments, the virus infection is an influenza virus. In some embodiments, the virus infection is a respiratory virus. The methods comprise intranasally administering to a human subject infected with a virus an effective amount of at least one antioxidant compound or a pharmaceutically acceptable salt thereof, wherein the method comprises administering a total daily dose of the antioxidant compound or salt thereof from about 0.001 to about 900 mg/kg. In some embodiments, the total daily dose is selected to provide a desired systemic level of the antioxidant compound or its metabolites in the subject. In some embodiments, the total daily dose is selected to provide a desired systemic level or localized level (such as in the brain or a part of the brain) of the antioxidant compound or its metabolites in the subject.

In some embodiments, the present methods comprise administering NAC or another antioxidant compound delivering NAC directly to the lungs. In some embodiments, the antioxidant compound is delivered directly to the bronchi of the lungs, such as the main bronchi, secondary bronchi, and/or tertiary bronchi. In some embodiments, the antioxidant compound is delivered directly to the bronchioles of the subject’s lungs. Delivering NAC directly to the lungs may allow a patient to leave the critical care unit or no longer need the support of a ventilator. In some embodiments, NAC treatment is used before or during treatment with a ventilator. It may also prevent a patient who does not have severe symptoms, from developing severe symptoms and needing to be moved into critical care or needing a ventilator.

In some embodiments, the antioxidant compound is administered in a manner that reduces irritation of the lung’s airways. There is a risk of bronchospasm when NAC is given by inhalation and to avoid this complication, NAC should be administered simultaneously with or following administration of an inhaled beta-adrenergic bronchodilator.

In some embodiments, the antioxidant compound is administered to a human subject at a total daily dose of from about 0.001 to about 900 mg/kg. The total daily dose can be administered on any desired schedule, such as once per day, twice per day, three times per day, four times per day, or more. The total daily dose can be divided into equivalent dosage amounts or differing dosage amounts, For instance, the dosage amount of the antioxidant compound can be approximately 150 mg/kg for an initial dose followed by one or more maintenance doses at dosage amounts of about 50 or 100 mg/kg. Maintenance doses can be repeated at appropriate intervals for a total of a desired number of doses. Total daily dose, dosage amounts and number of doses may be adjusted based on severity of COVID-19, so as to be less, the same, or more than the foregoing numbers.

U.S. Pat. Application No. 16/859,722, filed Apr. 27, 2020 and published as U.S. Pat. App. Publication No. 20200254073, discloses a small animal study that demonstrates delivering NAC intranasally and orally lead to an increase in GSH levels in the brain.

In some embodiments of the present methods, the delivery of NAC is targeted to the brain and/or the lungs acting on the coronavirus infection in each location. NAC is used for cystic fibrosis and has demonstrated benefits in disorders of the CNS, for instance in traumatic brain injuries. Some nasal delivery devices target the brain while others target the throat. Some may allow for administration to both the brain and the throat. Other routes of administration such as a nebulizer may target the lungs better, like how FDA-approved MUCOMYST is administered. In various suitable delivery devices, NAC may be sprayed as a mist or aerosolized. Suitable delivery devices may comprise a pump or a pressurized gas, and may be configured for a single use or for repeated or multiple uses. Oral administration of NAC may also provide benefits to the lungs, brain or both from complications due to coronavirus.

Intranasal (IN) delivery avoids first pass metabolism and may be able to bypass the blood brain barrier (BBB) when delivered via the nose. It may also be that the NAC does not enter the brain but is broken down to cysteine which crosses the blood brain barrier, which then leads to an increase of GSH in the brain. Regardless of the chemical or biological path, the present methods increase GSH in the brain and providing NAC can lead to this intended effect. Intranasal delivery may be the most efficient non-invasive way to get NAC (or a derivative of NAC) past the BBB, allowing for an increase in GSH. For extremely severe cases, it may be advantageous to apply NAC or glutathione intracranially to fight infections in the CNS. Intracranial administration of the antioxidant compound can be performed as described in McGavern U.S. Pat. No. 9,308,163 for administration of anti-inflammatory agents. McGavern discloses that intracranial application of glutathione after a traumatic brain injury, lead to a reduction in cell death.

In some embodiments, the present methods comprise administering GSH as the antioxidant compound or administering GSH and NAC together as the antioxidant compound.

The present methods can also comprise administering or formulating the antioxidant with one or more enhancers. There are several enhancers that may be able to help the delivery of NAC via nose-to-brain transport. Such nasal enhancers include but are not limited to cyclodextrins, such as (2-hydroxypropyl) beta-cyclodextrin (HPBCD). Another potential chemical that could expedite NAC’s delivery or its derivative to the brain is 1-O-n-dodecyl-B-maltopyranoside (DDM). Other compounds that can be used with NAC include glutathione, coenzyme Q-10, superoxide dismutase (SOD), and a combination thereof.

As another aspect, the present disclosure provides methods and apparatus to facilitate administration of an antioxidant compound by preventing or reducing unpleasant odors from the antioxidant compound. In some embodiments, a container comprising NAC or other antioxidant compound is a vacuum sealed container or vial. In some embodiments, a container comprising NAC or other antioxidant compound is a made by a method comprising storing NAC or other antioxidant compound under an inert atmosphere in such way that it prevents oxidation and/or filling the container in an inert atmosphere. A vial can be filled with an inert gas along with the NAC or another means to prevent the NAC from being exposed to oxygen, thereby preventing oxidation of NAC. It is believed the oxidation is what leads to the strong smell. In some embodiments, an intranasal delivery device is configured to load a vacuum sealed container containing NAC or other antioxidant agent. Most intranasally delivered drugs have their smell masked by other compounds or chemicals. The present intranasal delivery device is configured to load a vial of NAC or other antioxidant compound wherein the vial is vacuum sealed or prevented from mixing with oxygen, and delivering the NAC or other antioxidant compound to the subject’s nose, thereby preventing or reducing the odor associated with oxidation. This method may also increase the shelf-life of NAC or other antioxidant compound. The vial and delivery device to prevent oxidation is not limited to antioxidants and be applied to any therapeutic where oxygen is preferably avoided to increase shelf life or reduce smell.

Literature suggests that a vitamin D deficiency is leading to worse outcomes of COVID-19 patients. Data points to an increase in GSH levels being able to help decrease or prevent deficiencies of Vitamin D. In some embodiments of the present invention, an antioxidant compound is administered to a subject having a coronavirus infection at a dose and/or frequency to decrease or prevent deficiency of Vitamin D.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference in their entireties. The citation of any publication is for its disclosure prior to the filing date and should not be construed as an admission that the present claims are not entitled to antedate such publication. Further, the dates of publication provided can be different from the actual publication dates which can be independently confirmed.

As used in the specification and appended claims, the terms “a”, “an,” and “the” include both singular and plural referents, unless the context clearly dictates otherwise. Thus, for example, “a moiety” includes one moiety and plural moieties.

Any parts of the claims below can be used interchangeably with others.

### EXAMPLES

### Example 1

In this example, the efficacy of orally administered NAC was studied. The study was sponsored by the Cambridge Health Alliance and assigned Study ID NCT04419025. The purpose of this study was to assess the efficacy of N-acetylcysteine (NAC) in preventing those with mild or moderate COVID-19 from progressing to severe disease. Inclusion criteria for the study were known or suspect COVID-19 disease AND one or more of the following influenza-like symptoms, including: diarrhea vomiting fever (subjective or measured) chills myalgias fatigue sore throat headache cough nasal/sinus congestion or rhinorrhea shortness of breath chest pain.

N-acetylcysteine was administered as an oral formulation, as capsules containing 600 mg NAC. Participants were assigned to either an intervention group (those receiving NAC) or a control group (those not receiving NAC) for the duration of the study. Participants included inpatients and outpatients. Inpatients received NAC 25 mg/kg PO (taken by mouth) (rounded up to the nearest 600 mg) every 4 hrs until discharge, then NAC 1200 mg PO (taken by mouth) BID (twice a day) for 1 week post-discharge. Outpatients received NAC 2400 mg PO for one week, then 1200 mg PO BID for 2 weeks.

Preliminary data from the open label, controlled study of 160 randomized patients showed promising results for the treatment of subjects infected with SARS-CoV-2 and in need of treatment for COVID-19. The hospitalized group received 25 mg/kg every 4 hours and outpatient group received 2400 mg loading dose followed by 1200 mg, 2x/day for 2 weeks. All cases mild to moderate to start. The hospitalized group that received oral NAC had 1 less hospitalization day (from 6 days to 5 days) + no deaths. The hospitalized group that did not receive NAC had 2-3 deaths unfortunately.

For outpatients (all were mild cases to start), the NAC group had 3-4% needing hospitalization vs. 15% for those who did not get NAC. In summary, patients receiving oral NAC had 1 day less in the hospital and for those who were outpatient, there was roughly a 5x increase in hospitalization needed for those who did not receive oral NAC.

### Example 2

In this example, intranasal administration of NAC to healthy humans was studied. The small study was designed to determine the extent to which intranasal NAC leads to increased brain glutathione in healthy control subjects, as assessed using MEGA PRESS MR Spectroscopy.

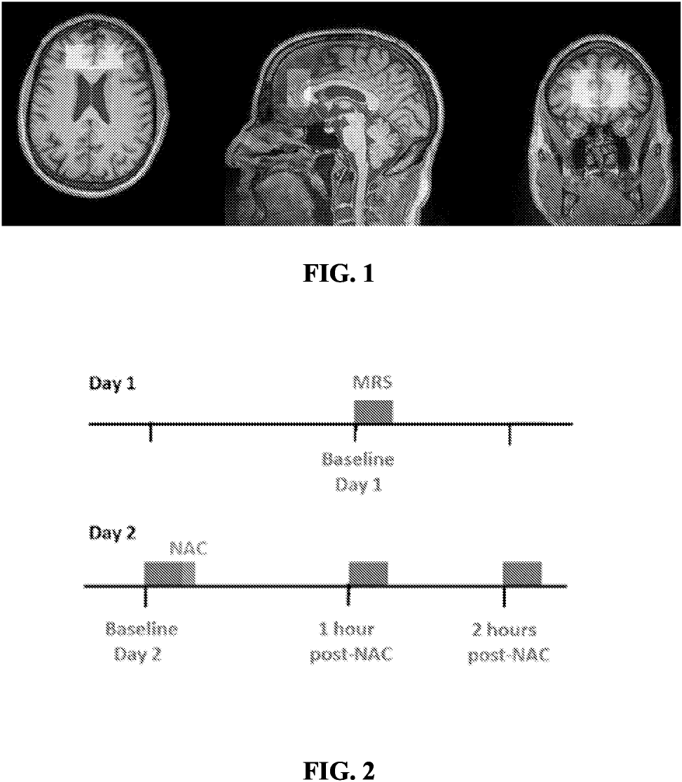

The study consisted of two study periods each involving 3 subjects and was designed to provide rapid proof-of-concept data on the ability of intranasal NAC to increase brain GSH levels. In Study Period 1, three right-handed male subjects, ages 21, 42, and 58 were evaluated. After informed consent and completion of a medical history questionnaire, each subject participated in multiple MR Spectroscopy sessions. On the first day, each subject was scanned once. A T1-weighted thin slice data set was acquired for subsequent positioning of a MR spectroscopy voxel (5×5×3 cm3) positioned with its posterior edge covering the genu of the corpus callosum, extending anteriorly into the frontal lobes. FIG. 1 shows images of the position of the MR spectroscopy voxel in the frontal lobes.

The cysteinyl β-CH2 of GSH exhibits a characteristic chemical shift at 2.95 p.p.m., which distinguishes it from other cysteine-based molecules. GSH levels were determined within the volume of interest using MEGAPRESS double-editing for the cysteinyl β-CH2 residue of GSH. Spectral editing was accomplished by refocusing GSH J-evolution during every other acquisition (ON), using a Gaussian pulse centered at the cysteinyl α-CH resonance of GSH at 4.56 p.p.m. During the alternate acquisitions (OFF), the pulse was applied symmetrically about the water peak. The difference-edited GSH spectrum was generated by subtraction of the OFF and ON spectra. Data analysis was accomplished using the GANNET software package which provided information on the GSH/Cr ratio. The Day 1 scan served as an initial baseline.

On Day 2, another baseline evaluation was performed, along with two additional scans at 1-hour and 2-hours post administration of intranasal NAC. The timing of Day 2 scans was shifted relative to Day 1, such that the time-of-day for the Day 1 baseline scan matched the time-of-day of the Day 2, 1-hour post NAC scan. This is illustrated in FIG. 2 and was done to provide control for potential circadian rhythm effects in GSH levels. In FIG. 2, green bars show times of spectroscopic imaging. The time-of-day for the baseline MRS scan on Day 1 matches the time-of-day for the 1-hour post-NAC scan on Day 2.

Intranasal NAC was delivered in the form of a 20% solution of MUCOMYST nasal spray, with 1 ml delivered to each nostril using a mucosal atomization device. Solution was delivered as six alternating bursts of 0.33 ml to each nostril over the course of 5-10 minutes.

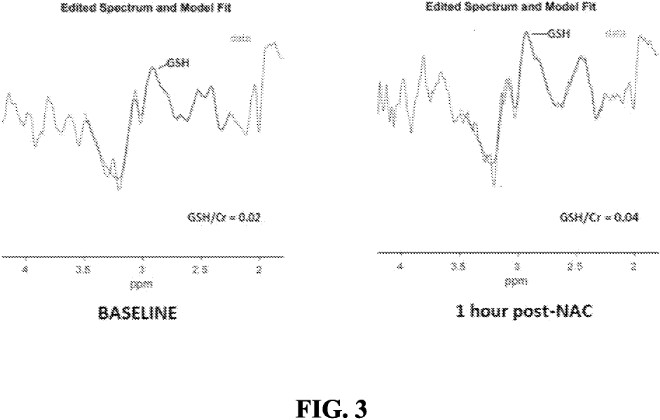

FIG. 3 shows results from Study period 1, and Table 1 provides GSH/Cr values at different time points in study period 1. FIG. 3 shows representative edited spectra for a single subject at Day 2 baseline and 1-hour post NAC scans. In looking at baseline values across subjects (shown in Table 1), there was considerable inter-individual variability, although values for a given subject were relatively stable across Day 1 and Day 2 baselines, and without evidence of an obvious circadian trend over the relatively short time-of-day window.

TABLE 1

| subject | Day 1, Baseline GSH/Cr | Day 2, Baseline GSH/Cr | Day 2, 1-hour post-NAC | Day 2, 2-hours post-NAC |
|---|---|---|---|---|
| 21 year old male | 0.042 | 0.038 | 0.059 | 0.052 |
| 42 year-old male | 0.017 | 0.020 | 0.037 | 0.035 |
| 58 year-old male | 0.026 | 0.024 | 0.031 | 0.048 |

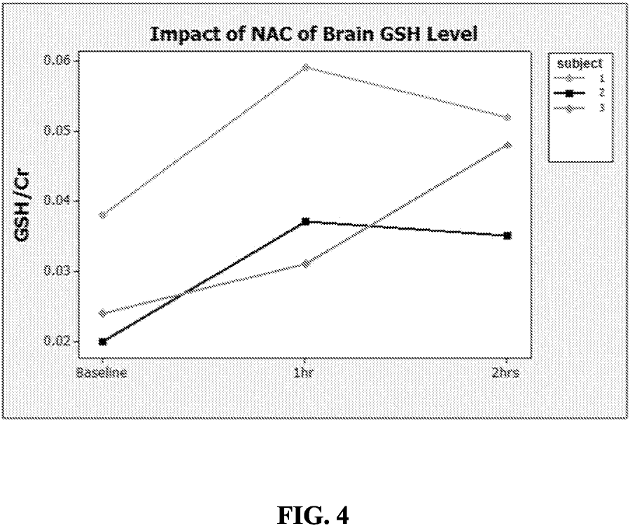

FIG. 4 illustrates the impact of Intranasal NAC on Brain GSH/Cr in the three study participants. The data demonstrates that intra-nasal NAC leads to a substantial increase in the brain GSH/Cr level that is sustained for at least 2 hours post-administration.

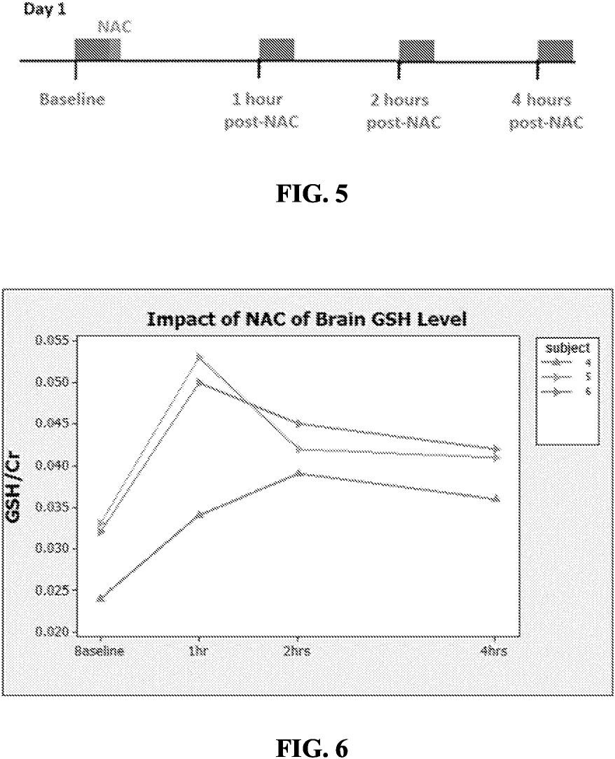

In Study Period 2, three additional right-handed male subjects, ages 23, 27, and 31 were studied. MRS data were collected on a single day, with baseline, 1 hr, 2 hrs, and 4 hrs post-NAC evaluations (as illustrated in FIG. 5). The same spectroscopic methods as used in period 1 were employed. However, a multi-dose pump intranasal delivery device was used.

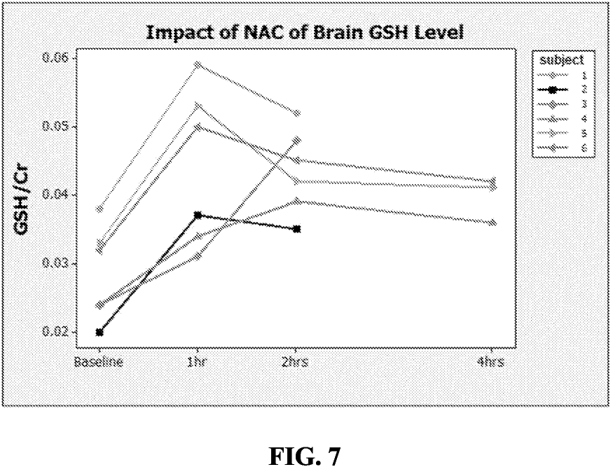

FIG. 6 and Table 2 provide the main results for study period 2. FIG. 6 illustrates the impact of Intranasal NAC on Brain GSH/Cr in the three participants in Study Period 2. Table 1 shows GSH/Cr values at different time points in study period 2.

TABLE 2

| subject | Baseline GSH/Cr | 1-hr post-NAC GSH/Cr | 2-hours post-NAC GSH/Cr | 4 hrs-hours post-NAC GSH/Cr |
|---|---|---|---|---|
| 23 year old male | 0.024 | 0.034 | 0.039 | 0.036 |
| 27 year-old male | 0.033 | 0.053 | 0.042 | 0.041 |
| 31 year-old male | 0.032 | 0.050 | 0.045 | 0.042 |

As was seen in study period 1, intranasal administration of NAC results in a substantial increase in brain GSH/Cr levels. Importantly, the data indicate that increased levels are maintained for at least 4 hrs, with only a slow rate of decay beyond hour 2. This suggests that 2x daily dosing of intranasal NAC will be sufficient to maintain increased GSH levels.

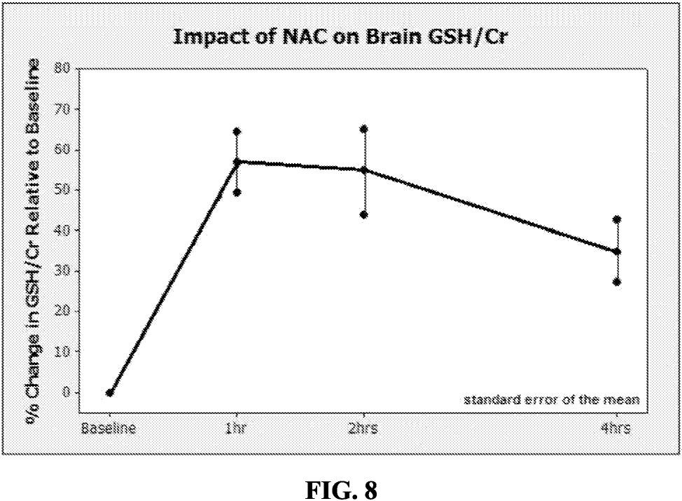

FIG. 7 shows spectroscopic data from all the individual subjects, with FIG. 8 showing the time course for the average percent change in GSH/Cr combined across all subjects. Subjects 1-3 were from study period 1 only measuring out to 2 hours, while subjects 4-6 were from study period 2, measured out to 4 hours. FIG. 8 shows that intranasal NAC leads to a substantial increase (>50%) in brain GSH levels and even at 4 hours, levels are ~35% above baseline. Error bars are SEM. The data demonstrate a clear peak in NAC levels between 1-2 hours with sustained levels at 4 hours. At each post-NAC time point, the change in GSH/Cr level relative to baseline was statistically significant (1 hr < p.001; 2 hours P < .005; 4 hours p < .05).

With respect to safety, no adverse events were reported by any subjects. No significant changes in heart-rate, breathing rate, O2 saturation or blood pressure were seen. No significant side-effects were seen although 5 of 6 subjects did voice complaints about the NAC odor, and 2 reported very mild transient nausea (< 5 minutes). For the mucosal atomizer, all three subjects complained of NAC fluid into the throat. This was not found with the other nasal delivery device (the multi-dose pump).

This study provides initial evidence that intranasal NAC leads to a statistically significant increase in brain GSH as measured by MR spectroscopy.

### Example 3

This example describes physiological based pharmacokinetic modeling and simulation (PBPK) that provides evidence of significantly increased bioavailability of NAC due to intranasal (IN) administration. The analysis was also used to model the impact of an enhancer of paracellular movement on IN administration of NAC.

The PBPK analysis used naltrexone and sumatriptan for comparison purposes. Table 3 summarizes the PBPK analysis, with values for bioavailability (“F”) as a percentage of the dose administered. For naltrexone and sumatriptan, bioavailability measurements were available as a means of assessing the predictive ability of the model.

TABLE 3

| Drug | Dose (mg) | F total meas (%) | F total pred (%) | F intra nasal (%) |
|---|---|---|---|---|
| Naltrexone | 4 | 65 | 69 | 65 |
| Naltrexone | 4 + DDM | 95 | 98 | 97 |
| Sumatriptan | 20 | 34 | 75 | 72 |
| Sumatriptan¹ | 20 | 34 | 30 | 27 |
| Sumatriptan | 10 + DDM | 69 | 64 | 60 |
| NAC² | 20 | - | 92 | 91 |
| NAC | 20 + DDM | - | 99 | 98 |

F total meas= total bioavailability experimentally determined
F total pred= total bioavailability predicted by G+
F intranasal= intranasal part of total predicted bioavailability
1 - assuming metabolism in nasal tissue
2 - assuming no metabolism in nasal tissue

Both Naltrexone and Sumatriptan IN absorption increased when dosed together with DDM, resulting in an increase in bioavailability of 30% and 35%, respectively. NAC is predicted to have high intranasal absorption, unless there is metabolism in nasal tissue; if metabolism occurs, the use of DDM or another enhancer will likely reduce this.

The PBPK analysis suggests that by avoiding the first pass metabolism seen with oral dosing, IN administration can increase bioavailability to 90% from 10% oral dosing. The model predicts that with a 30fold decrease in the amount of drug delivered IN vs oral, there is only a ~40% drop in the peak concentration in circulation and the time to this peak is much faster, (~10 min IN versus 1 hour oral). This pharmacokinetic profile would be especially beneficial for treating a subject infected with SARS-CoV-2 or in need of treatment for COVID-19, since it would be desirable to have the drug in blood circulation to reach lung tissue.

It is projected that IN NAC will increase glutathione levels faster than oral NAC and help COVID-19 patients more. The bioavailability of oral NAC is around 9-10% whereas based on a PBPK analysis of N-acetyl cysteine, the predicted bioavailability of intranasal NAC is just over 90%. Using an enhancer suggests a bioavailability of up to 100%, the same as IV.

It is unexpected that intranasal administration of NAC could yield a bioavailability the same as, or even close to, intravenous administration. Intranasal administration has several advantages over IV administration, which is costly and requires hospitalization. Having access to intranasal NAC is ideal due to its ease of administration, low or no side effects and low cost.

1. A method of treating a disease or disorder associated with a coronavirus infection, the method comprising:
intranasally administering to a human subject infected with a coronavirus an effective amount of at least one antioxidant compound or a pharmaceutically acceptable salt thereof, wherein the method comprises administering a total daily dose of the antioxidant compound or salt thereof from about 0.001 to about 900 mg/kg.

2. The method of claim 1, wherein the human subject is infected with SARS-CoV-2.

3. The method of claim 1, wherein the human subject is in need of treatment for COVID-19.

4. The method of claim 1, wherein the antioxidant compound is administered at a dose effective to loosen sputum in the lungs of the human subject.

5. The method of claim 4, wherein the dose is additionally effective to elevate GSH level in the brain of the human subject.

6. The method of claim 1, wherein the antioxidant compound is selected from the group consisting of N-acetylcysteine (NAC), glutathione, co-enzyme Q-10, superoxide dismutase (SOD), and a combination thereof.

7. The method of claim 1, wherein the method further comprises administering to the subject matrix metallopeptidase 9 (MMP-9) or a biologically active fragment or variant thereof.

8. The method of claim 1, wherein the method further comprises administering to the subject a non-steroid anti-inflammatory agent.

9. The method of claim 1, wherein the method further comprises administering to the subject trofinetide, progesterone, neurosteroid, a ghrelin compound, a salt thereof, or a combination thereof.

10. The method of claim 1, wherein the disease or disorder associated with a coronavirus infection is a central nervous system disease.

11. The method of claim 10, wherein the CNS disease is selected from: stroke, dizziness, headache, seizures, impaired consciousness, acute cerebrovascular problems, cerebral hemorrhage, confusion, direct brain infection and injury, toxic-metabolic encephalopathy, demyelinating disease, anosmia, and ageusia.

12. The method of claim 1, wherein the method further comprises administering to the subject an agent for enhancing delivery of and/or alleviating odor from the at least one antioxidant compound or salt thereof.

13. The method of claim 12, wherein the agent comprises a cyclodextrin compound in an amount effective to enhance delivery of and/or alleviate odor from the at least one antioxidant compound or salt thereof.

14. The method of claim 12, wherein the agent comprises (2-hydroxypropyl) beta-cyclodextrin (HPBCD).

15. The method of claim 12, where the agent comprises 1-O-n-dodecyl-B-maltopyranoside (DDM).

16. The method of claim 1, wherein the at least one antioxidant compound or salt thereof is administered as a pharmaceutical formulation comprising one or more sweetening, flavoring or perfuming agents.

17. The method of claim 1, wherein the at least one antioxidant compound or salt thereof is administered as a pharmaceutical formulation comprising one or more additives that prevents or slows oxidation of the at least one antioxidant compound or salt thereof.

18. The method of claim 1, wherein the at least one antioxidant compound or salt thereof is administered from 1 to 365 days.

19. The method of claim 1, wherein the at least one antioxidant compound or salt thereof is used as a prophylactic.

20. The method of claim 1, wherein at least one antioxidant compound or salt thereof is administered to a human having mild symptoms, or before treatment with a ventilator.

21-32. (canceled)
